(19) United States
(12) Patent Application Publication
(10) Pub. No.: US 2008/0269610 A1
(43) Pub. Date: Oct. 30, 2008

(54) Method and apparatus for automatic optimization of scanning parameters for ultrasound imaging

(75) Inventors: Elina Burla, Kiryat Tivon (IL); Alexander Sokulin, Kiryat Tivon (IL); Doron Hess, Haifa (IL); Eran Gitlin, Nesher (IL)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY

(21) Appl. No.: 11/789,788

(22) Filed: Apr. 25, 2007

(51) Int. Cl. A61B 8/00

(52) U.S. Cl. 600/447

(57) ABSTRACT

An ultrasonic probe has transducer elements for transmitting and receiving ultrasound signals to form an ultrasound image. An optimization module receives a first scanning depth based on the ultrasound image and determines a first transmit pattern and receive filter settings based on the first scanning depth. The first transmit pattern and the receive filter settings are used to acquire the ultrasound image. Transmit and receive circuitry transmit and receive the ultrasound signals based on the first transmit pattern

### BACKGROUND OF THE INVENTION

This invention relates generally to ultrasound imaging, and more particularly, to automatically optimizing scanning parameters.

Resolution and penetration are two main measures of quality in ultrasound imaging. Ultrasound spatial resolution is defined as the minimum distance at which two adjacent reflecting objects can be distinguished. Spatial resolution can be improved, in general, by increasing transmit frequency and beam-density. Ultrasound penetration is defined as the maximum depth from which usable signals return to the transducer. The penetration depth can be increased to view deeper structures by decreasing the transmit frequency. Therefore, the choice of frequency is a trade-off between the image spatial resolution and the penetration depth.

An operator may also change the beam-density, which is a trade-off between frame-rate and lateral resolution. Increasing the beam-density increases the lateral resolution up to an upper limit level that is a function of beam-width and pixel density of the display-raster of a display used to display the ultrasound image.

Currently, broadband ultrasound probes are available that provide a wide spectrum of transmit frequencies within a single probe. Thus, the same probe may be used to image a wide range of patients and/or anatomical structures. During an ultrasound examination, the operator has to balance the technical compromise of resolution versus penetration. An operator often begins a routine scanning procedure by visualizing structures that lie proximal to the probe by using a minimal depth and a higher frequency to provide the best resolution. To view deeper structures the operator increases the depth of the field of view and then manually tunes the transmit frequency to improve the image quality. In some systems, the ultrasound system may automatically adjust the transmit frequency based on the new depth. This automatic adjustment may “de-tune” selections made by the operator specific to the patient.

The operator may change (increase and decrease) the depth at various times throughout the exam. Manual tuning of the transmit frequency is time consuming and does not always result in optimal image quality as the operator may forget to adjust the frequency and/or select an incorrect frequency. For example, lack of information and increased noise in the far field result when depth is increased and the frequency is not adjusted properly. Also, poor resolution results when the depth is decreased and the frequency is not adjusted properly.

In some cases, a predetermined combination of scanning parameters, such as a depth, associated transmit frequency and beam-density (frame-rate) may be stored in an application preset that is selectable by the operator through a preset button or other selectable device. The application presets do not take into account differences in patients and anatomy, and thus the operator may still need to optimize scanning parameters after selecting the application preset. When the operator subsequently changes the depth or transmit frequency during the procedure, the scanning parameters may again need to be manually optimized.

Therefore, a need exists for a system that provides automatic adjustment of the scanning parameters to optimize scanning and that also preserves operator optimizations, if present.

### BRIEF DESCRIPTION OF THE INVENTION

In one embodiment, an ultrasonic probe has transducer elements for transmitting and receiving ultrasound signals to form an ultrasound image. An optimization module receives a first scanning depth based on the ultrasound image and determines a first transmit pattern and receive filter settings based on the first scanning depth. The first transmit pattern and the receive filter settings are used to acquire the ultrasound image. Transmit and receive circuitry transmit and receive the ultrasound signals based on the first transmit pattern.

In another embodiment, a method for optimizing an ultrasound image comprises scanning with a first transmit pattern based on a first scanning depth to acquire an ultrasound image in a first scanning mode. A second scanning depth is received that is different with respect to the first scanning depth. A second transmit pattern is selected based on the second scanning depth, the first and second transmit patterns being different with respect to each other.

In yet another embodiment, a method for optimizing an ultrasound image comprises selecting a first transmit pattern based on a first scanning depth within an ultrasound image. The first transmit pattern further defines values for at least one of pulse sequence, shape of pulses, number of pulses, amplitude of pulses, and transmit frequency. A first beam-density is selected based on at least the first scanning depth. A second scanning depth is received that is different with respect to the first scanning depth. A second transmit pattern and a second beam-density are selected based on the second scanning depth wherein the second transmit pattern is different with respect to the first transmit pattern.

### BRIEF DESCRIPTION OF THE DRAWINGS

FIG. 1 illustrates a block diagram of an ultrasound system formed in accordance with an embodiment of the present invention.

FIG. 2 illustrates a miniaturized ultrasound system formed in accordance with an embodiment of the present invention.

FIG. 3 illustrates an example of optimal scanning parameters that may be stored within a memory of an ultrasound system in accordance with an embodiment of the present invention.

FIG. 4 illustrates a first image that comprises ultrasound data associated with a first scanning depth in accordance with an embodiment of the present invention

FIG. 5 illustrates a second image that comprises ultrasound data associated with a second scanning depth in accordance with an embodiment of the present invention.

FIG. 6 illustrates a method for automatically optimizing scanning parameters for acquiring an ultrasound image in accordance with an embodiment of the present invention.

FIG. 7 illustrates an example of a series, family or set of optimal curves that represent optimal transmit patterns as a function of scanning depth and/or transmit frequency in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

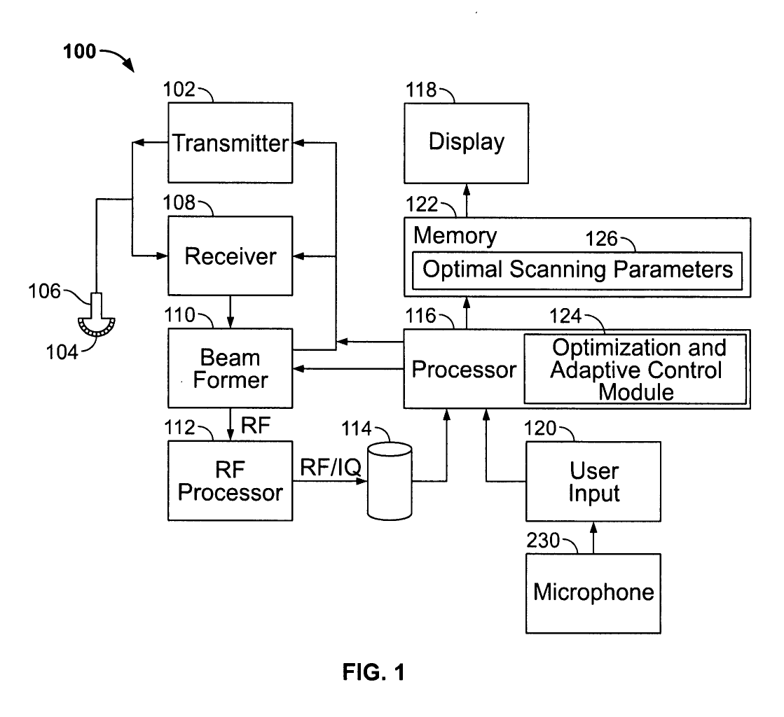

FIG. 1 illustrates a block diagram of an ultrasound system 100. The ultrasound system 100 includes a transmitter 102 that drives transducer elements 104 within a probe 106 to emit pulsed ultrasonic signals into a body. The transmitter 102 transmits the ultrasonic signals in a transmit pattern that is further discussed below. A variety of probe geometries may be used. The ultrasonic signals are back-scattered from structures in the body, like blood cells or muscular tissue, to produce echoes that return to the transducer elements 104. The returning echoes are converted back to electrical energy by the transducer elements 104 which are received by a receiver 108. The received signals are passed through a beamformer 110 that performs beamforming (combining the transducer element signals to perform steering and focusing of the beam) and outputs an RF signal. The receiver 108 and/or beamformer 110 may process the received signals with receive filter settings that are based at least on a defined depth. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to an RF/IQ buffer 114 for temporary storage.

A user input 120 may be used to control operation of the ultrasound system 100, including, to control the input of patient data, scan parameters, change scanning parameters such as transmit pattern, transmit frequency, change a depth parameter, and the like, and may also include using voice commands provided via a microphone 230. Other various embodiments such as a set of user controls may be configured for controlling the ultrasound system 100 and may be provided, for example, as part of a touch screen or panel, and as manual inputs, such as user operable switches, buttons, and the like. The set of user controls may be manually operable or voice operated.

The ultrasound system 100 also includes a processor 116 to process the acquired ultrasound information (i.e., RF signal data or IQ data pairs) and prepare frames of ultrasound information for display on display 118. The display 118 has a known resolution that may be defined in terms of pixels or other known parameter. The processor 116 is adapted to perform one or more processing operations according to a plurality of selectable ultrasound modalities on the acquired ultrasound information. Acquired ultrasound information may be processed in real-time during a scanning session as the echo signals are received.

An optimization and adaptive control module 124 (hereafter optimization module 124) may access optimal scanning parameters 126 stored within a memory 122 to automatically control the scanning parameters. The optimization module 124 and optimal scanning parameters 126 may be implemented in hardware or software, or a combination thereof. The optimization module 124 may automatically detect or receive a scanning depth as an input parameter. For example, when a probe 106 is first connected to the system 100 or when the operator selects a preset or a particular application protocol, the system 100 may set a depth parameter at a predetermined initial depth setting that is provided to the optimization module 124 as an input parameter. The operator may manually control the scanning depth by using the user input 120 to increase and decrease the scanning depth. Any operator change to the depth parameter is also provided to the optimization module 124. The scanning depth may identify the penetration depth of a scanning image and/or a depth of a subset of the scanning image, such as the depth of a range gate or region of interest (ROI). The optimization module 124 may automatically optimize scanning parameters based on the depth, such as by accessing predefined tables, charts, databases, formulas, and/or other mapping functionality stored within the optimal scanning parameters 126. The optimization may also change based on one or more of the type of probe 106 being used, an operational mode of the system 100 and/or a scanning protocol being used.

The optimization module 124 also provides adaptive control to reflect and thus preserve operator changes to scanning parameters such as the transmit frequency. For example, while the ultrasound system 100 may typically assume a constant acoustic velocity of 1540 m/s, in practice the ultrasound energy is influenced by the acoustical impedance of the patient, which may vary from patient to patient and from one anatomical structure to the next. Therefore, customizations based on operator input, such as to reflect specific patient characteristics and/or operator preference, may automatically be applied even when the depth is further changed.

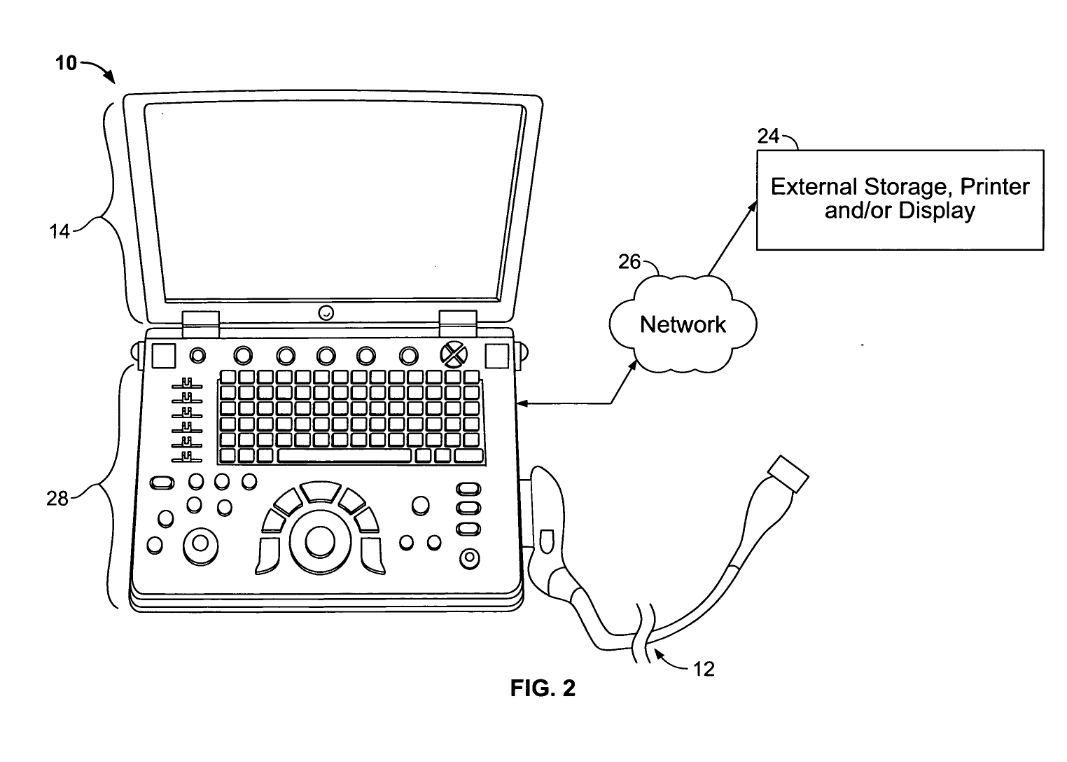

It should be understood that the functionality discussed with respect to the system 100 is not limited to any ultrasound system type. For example, the system 100 may be housed within a cart-based system or may be implemented in a smaller, portable system as discussed in FIG. 2.

FIG. 2 illustrates a miniaturized ultrasound system 10 having a probe 12 configured to acquire ultrasonic data. As used herein, “miniaturized” means that the ultrasound system is a handheld or hand-carried device or is configured to be carried in a person's hand, pocket, briefcase-sized case, or backpack. For example, the ultrasound system 100 may be a hand-carried device having a size of a typical laptop computer, for instance, having dimensions of approximately 2.5 inches in depth, approximately 14 inches in width, and approximately 12 inches in height. The ultrasound system 100 may weigh about ten pounds, and thus is easily portable by the operator. An integrated display 14 (e.g., an internal display) is also provided and is configured to display a medical image.

The ultrasonic data may be sent to external device 24 via a wired or wireless network 26 (or direct connection, for example, via a serial or parallel cable or USB port). In some embodiments, external device 24 may be a computer or a workstation having a display. Alternatively, external device 24 may be a separate external display or a printer capable of receiving image data from the hand carried ultrasound system 10 and of displaying or printing images that may have greater resolution than the integrated display 14. The integrated display 14 and external device 24 may each have a different resolution that is known to the ultrasound system 10.

A user interface 28 (that may also include integrated display 14) is provided to receive commands from an operator, such as to change the scanning depth. Scanning parameters may be modified based on the resolution of the integrated display 14, on a different external display 24, or based on an operator defined resolution. Therefore, the acquired image data may be acquired in a higher resolution than that displayable on integrated display 14.

As another example, the ultrasound system 10 may be a pocket-sized ultrasound system. By way of example, the pocket-sized ultrasound system may be approximately 2 inches wide, approximately 4 inches in length, and approximately 0.5 inches in depth and weigh less than 3 ounces. The pocket-sized ultrasound system may include a display, a user interface (i.e., keyboard) and an input/output (I/O) port for connection to the probe (all not shown). It should be noted that the various embodiments may be implemented in connection with a miniaturized ultrasound system having different dimensions, weights, and power consumption.

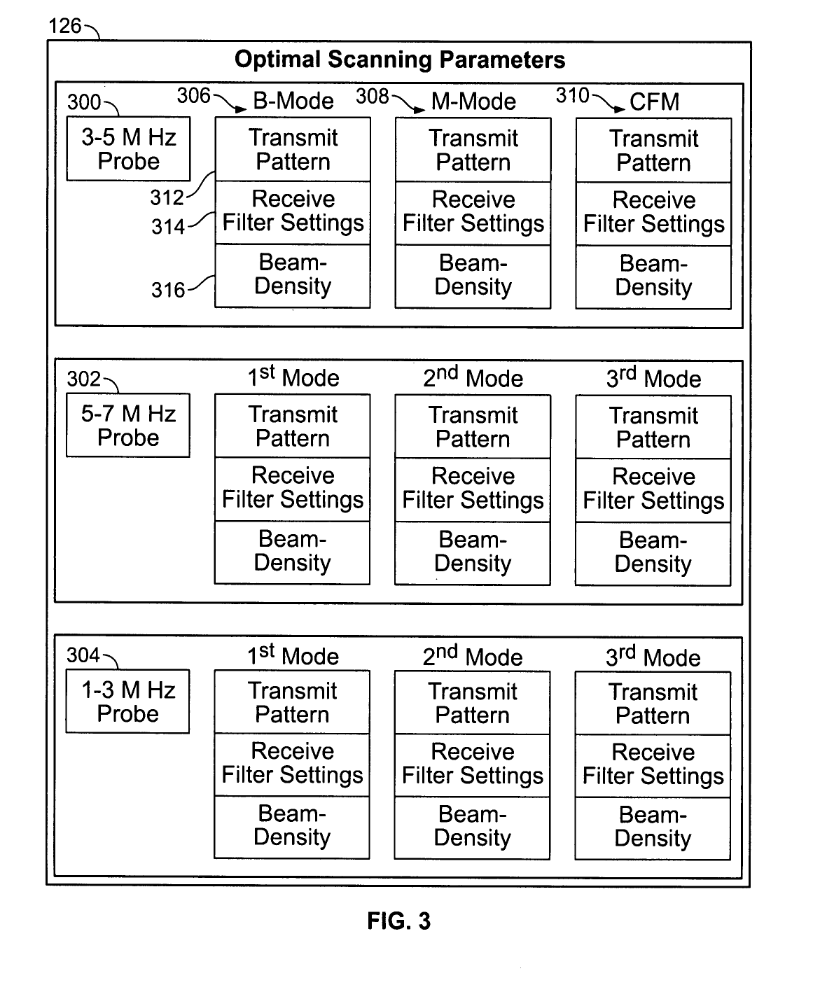

FIG. 3 illustrates an example of optimal scanning parameters 126 that may be stored within the memory 122 (shown in FIG. 1). Optimal scanning parameters 126 may be determined for each of a plurality of different probes, such as a 3-5 MHz probe 300, a 5-7 MHz probe 302 and a 1-3 MHz probe 304. For each of the probes 300-304, at least one set of optimal scanning parameters may be determined and multiple sets of optimal scanning parameters may be determined for different operational modes. It should be noted that not all of the probes 300-304 may be configured to operate in each of the modes, and that not all possible probes types and operational or scanning modes are illustrated in FIG. 3. Generally, for each different probe, each mode may have a different set of scanning parameters, although overlap may exist.

For the 3-5 MHz probe 300, three scanning modes or modes of operation are indicated as B-mode 306, M-mode 308 and Color Flow Mode (CFM) 310. For each mode 306-310, an optimal transmit pattern, receive filters and beam-density are identified. For example, optimal transmit pattern 312, receive filters settings 314 and beam-density 316 are identified for the 3-5 MHz probe 300 operating in the B-mode 306. The optimal transmit pattern 312 may comprise one or more parameters such as pulse sequence, shape of pulses, number of pulses, amplitude of pulses, and transmit frequency. Other parameters may be defined within the optimal transmit pattern 312. As discussed further below, the beam-density 316 may be further based on a display resolution or an operator defined resolution.

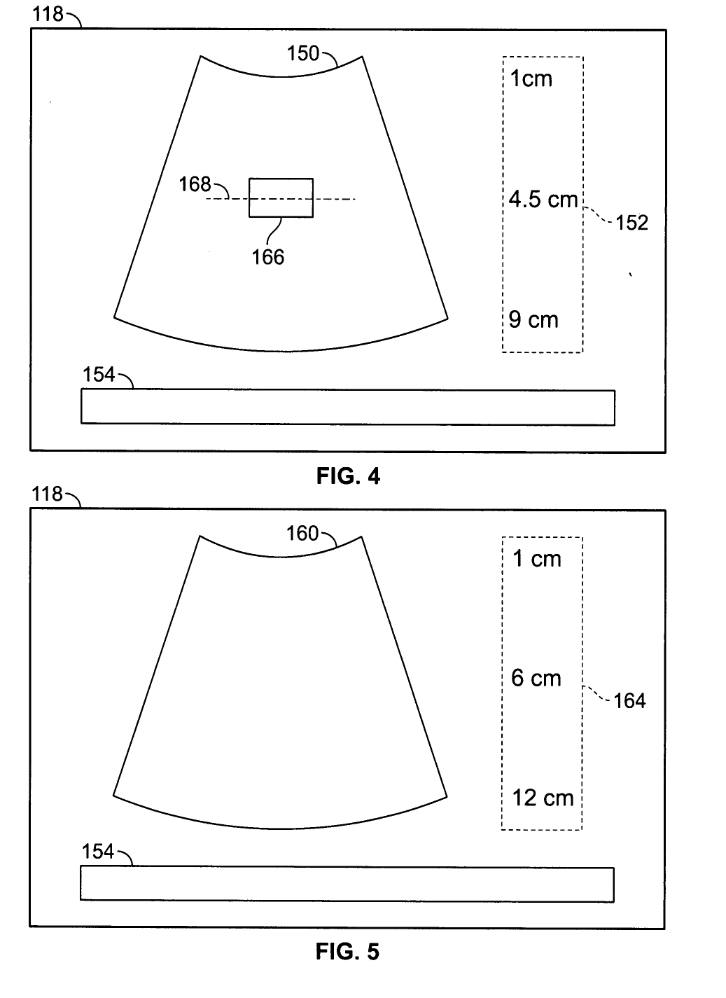

FIGS. 4 and 5 illustrate first and second images 150 and 160 that comprise ultrasound data having different scanning depths that may be acquired by the probe 106. The first image 150 has a first depth 152 of approximately 9 cm while the second image 160 has a second depth 164 of approximately 12 cm. In this example, the scanning depth parameter of the first and second depth 152 and 164 refers to the penetration depth of the field of view (FOV) of the ultrasound image (e.g. first and second images 150 and 160) that may be displayed on the display 118 of FIG. 1 and integrated display 14 of FIG. 2. The values for the first and second depth 152 and 164 may be displayed proximate to the first and second images 150 and 160 for reference. Optionally, the values for the first and second depth 152 and 164 may be displayed in a different location, such as along an edge region 154 of the display 118. Other scanning parameters, such as scanning mode (e.g. B-mode, CFM, M-mode, pulsed-wave (PW) Doppler mode, and the like) and transmit frequency, as well as other scanning parameters that are used to form the first image 150, may also be displayed on the display 118, such as within the edge region 154 or as an overlay over or proximate to the first image 150.

In another embodiment, the depth may be determined relative to a range gate or region of interest (ROI) 166, such as along a center 168 of the ROI 166 as illustrated on the first image 150. For example, if Doppler is used, a range gate may be defined and if CFM mode is used the ROI 166 may be defined.

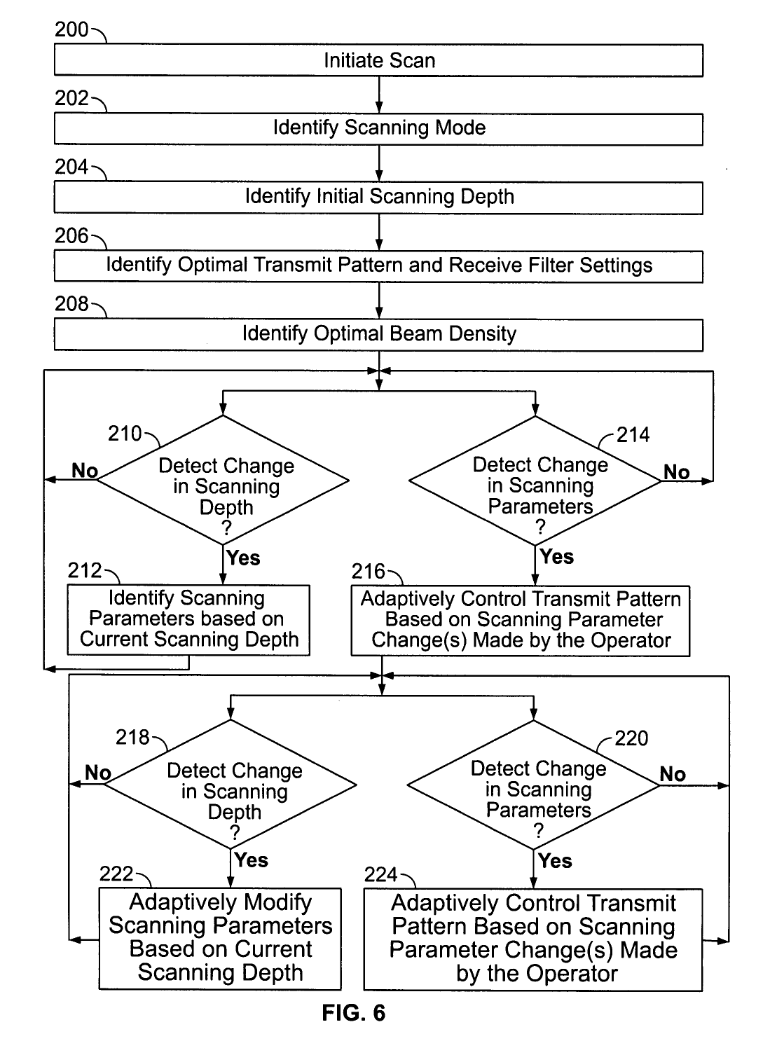

FIG. 6 illustrates a method for automatically optimizing scanning parameters for acquiring an ultrasound image. At 200, an operator initiates an ultrasound scan using the system 100 or 10 of FIGS. 1 and 2, respectively, and the probe 106 or 12, respectively. For simplicity, the system 100 and components thereof will primarily be discussed below. The operator may choose the type of probe 106 based on exam properties or requirements or on the scanning mode to be used. At 202, the operator may identify a scanning mode, such as by entering the data manually or by selecting a preset or predetermined protocol. As discussed previously, the scanning mode may be one of B-mode, CFM, M-mode, PW Doppler mode, or other imaging mode known in the art. The scanning mode is not limited to those discussed herein.

At 204, the optimization module 124 identifies an initial scanning depth, such as by receiving an operator input that selects the initial scanning depth and/or by receiving a scanning depth parameter from the processor 116. The optimization module 124 may also retrieve the initial scanning depth, such as from a predetermined location in the memory 122. The scanning depth may be a penetration depth or a depth defined relative to the ROI 166, such as at the center 168 as is illustrated in FIG. 4. As discussed previously, the initial scanning depth may also be a preset value determined by a protocol, an application preset, a default position of the ROI 166, and/or associated with the type of probe 106 being used.

Optimal scanning parameters are then determined to acquire the first image 150 at the initial depth. At 206, the optimization module 124 may determine an optimal transmit pattern that defines one or more of pulse sequence, shape of pulses, number of pulses, amplitude of pulses, and transmit frequency as discussed previously. The optimization module 124 may also determine optimal receive filter settings (used by the receiver 108 and/or the beamformer 110). Referring to FIG. 3, if the probe 106 is the 3-5 MHz probe 300 acquiring data in B-mode 306, the optimal transmit pattern 312 and receive filters settings 314 may be applied.

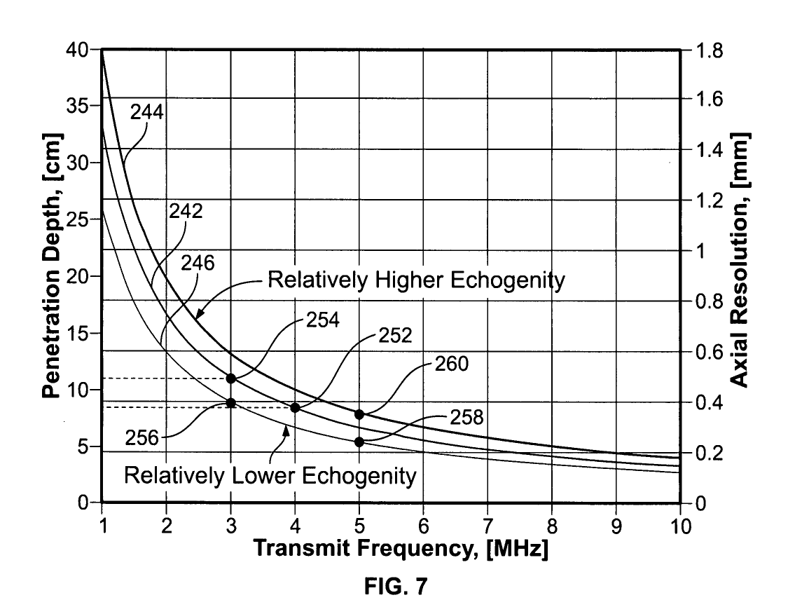

FIG. 7 illustrates an example of a series, family or set of optimal curves 240, each of which represents an optimal transmit pattern as a function of scanning depth and/or transmit frequency. The set of optimal curves 240 may comprise a plurality of curves such as first optimal curve 242, second optimal curve 244 above the first optimal curve 242 and third optimal curve 246 below the first optimal curve 242. Although three optimal curves are shown, it should be understood that a set of optimal curves may comprise more or less than three optimal curves. The first optimal curve 242 may represent a standard or average echnogenity, the second optimal curve 244 may represent a relatively high echogenity with respect to the first optimal curve 242, and the third optimal curve 246 may represent a relatively low echogenity with respect to the first optimal curve 242. Each of the first through third optimal curves 242-246 represents a set of parameters that may be optimal for a different type of patient and/or anatomy. By way of example, the set of optimal curves 240 may be applicable for a probe 106 having a frequency range from 3-5 MHz, such as the probe 300 of FIG. 3. Also, the set of optimal curves 240 may be applicable for a particular imaging mode, such as B-mode 306, and the optimal transmit pattern 312 may be based on one of the first through third optimal curves 242-246. Other probes having other frequency ranges may have their own set of optimal curves, and additional curves may exist for each imaging mode.

Returning to 206 of FIG. 6, the optimization module 124 may identify the first optimal curve 242 which may be based on previous studies or representative of a large percentage of patient data. Optionally, the first optimal curve 242 may be the mean or average curve of the set of optimal curves 240. In an example, first point 252 may be defined on the first optimal curve 242 based on the first depth 152 of approximately 9 cm of the first image 150 of FIG. 4. Based on the first point 252, a transmit frequency (one of the components of the optimal transmit pattern 312) of 4 MHz may be selected.

At 208, the optimization module 124 may identify an optimal beam-density 316 and/or or frame-rate. The beam-density 316 may be selected based on both the scanning depth (at 204) and the resolution of one or more displays 118 interconnected with the system 100. For example, the beam-density 316 may be selected based on the pixel resolution of the display 118 such that the beam or line density results in an image having a resolution that is approximate to or not much higher than the resolution of the display 118. Optionally, the beam-density 316 may be determined by the operator defined resolution and/or by an external display or device, such as the external device 24 of FIG. 2, in which case the beam-density 316 may be greater than can be displayed on the integrated display 14. For example, it may be desirable to acquire data at a greater beam-density to save, display and/or print, simultaneous with the acquisition or later, at a resolution higher than the resolution of the integrated display 14.

The optimal scanning parameters identified at 206 and 208 are used to acquire imaging data until the scanning depth is changed or the operator manually changes a scanning parameter, such as through the user input 120. For example, the operator may optionally modify or change scanning parameters or parameters of the transmit pattern, such as the transmit frequency, select different receive filters that are applied by the receiver 108 and/or beamformer 110, and/or change the beam-density or frame-rate to modify the first image 150. The scanning depth change will be discussed first.

At 210, if the system 100 detects that the operator has changed the scanning depth, the method continues to 212. For example, the first depth 152 may be manually changed by the operator, such as by turning a knob or activating another device on the user input 120. As discussed previously, the scanning depth change may be a change in penetration depth or a depth of the ROI 166 or sample gate. Turning to FIG. 5, the second image 160 has a greater penetration depth with respect to the first image 150 of FIG. 4. In this example, the first depth 152 has been increased to a second depth 164 of 12 cm to allow the operator to view anatomical structures located further from the probe 106.

Returning to FIG. 6, at 212 the optimization module 124 identifies an optimal transmit pattern 312 for the current scanning depth. In the example above, the current scanning depth is 12 cm. The optimization module 124 moves along the first optimal curve 242 (of FIG. 7) from the first point 252 to a second point 254 that corresponds to 12 cm in depth. The transmit frequency may now be approximately 3 MHz. Other parameters within the optimal transmit pattern 312 may also change, as well as the receive filter settings 314. In one embodiment, the optimization module 124 may access data within the optimal scanning parameters 126, such as a chart, database, configuration file(s) and the like, to determine new parameters for one or more parameters within the optimal transmit pattern 312 based on the current scanning depth. In another embodiment, if the current scanning depth is reflecting a change in the depth of the center 168 of the ROI 166 (FIG. 4), the optimization module 124 will also identify an optimal transmit pattern 312 by moving along the current optimal curve. The beam-density 316 may also be changed based on the current scanning depth

At 214, if the optimization module 124 determines that the operator has changed a scanning parameter other than the scanning depth, such as the transmit frequency, the method continues to 216 where the optimization module 124 adaptively controls the optimal transmit pattern 312 based on the change made by the operator. If the transmit frequency is changed, the optimization module 124 moves to a different optimal curve within the set of optimal curves 240. By way of example and not limitation, when the operator decreases the transmit frequency, the optimization module 124 selects a lower curve with respect to the current curve, performing adaptation to lower patient echogenity. When the operator increases the transmit frequency, the optimization module 124 selects a higher curve with respect to the current curve, performing adaptation to higher patient echogenity. For example, assuming a current setting at the first point 252, the operator may change the transmit frequency from approximately 4 MHz to approximately 3 MHz without changing the depth. The optimization module 124 selects a third point 256 on the third optimal curve 246. The optimal transmit pattern 312 is now based on the third point 256 and the third optimal curve 246.

Once the operator has changed a scanning parameter, the system 100 will adaptively determine any further scanning changes based on the operator changes. At 218 and 220, the optimization module 124 monitors to determine whether a change in scanning depth or a change in a scanning parameter, respectively, has been made by the operator. If a change in scanning depth is detected at 218, at 222 the optimization module 124 may access the optimal scanning parameters 126 to identify an optimal transmit pattern 312, receive filter settings 314 and beam-density 316 based on the current scanning depth. For example, assuming a starting point of the third point 256 located on the third optimal curve 246, if the operator changes the depth from approximately 9.5 cm to approximately 6 cm the optimization module 124 moves along the third optimal curve 246 to fourth point 258. Therefore, the previous modifications the operator made to the scanning parameters have been adaptively taken into consideration when adjusting the optimal transmit pattern 312.

If a change to a scanning parameter is detected at 220, the method continues to 224 where the optimization module 124 adaptively controls the optimal transmit pattern 312 based on the scanning parameter change. For example, assuming a starting point of the third point 256 located on the third optimal curve 246, if the operator changes the transmit frequency from approximately 3 MHz to approximately 5 MHz, the optimization module 124 may select a fifth point 260 along the second optimal curve 244. The optimization module 124 identifies an optimal transmit pattern 312 based on the fifth point 260, as well as optimal receive filter settings 314 and beam-density 316. Therefore, changes the operator has made to the scanning parameters are adaptively reflected in the optimal transmit pattern 312, and the optimal transmit pattern 312 is optimized based on the operator changes that were made to reflect the particular patient's anatomical composition, structure being imaged, operator preference, and the like.

A technical effect of at least one embodiment is automatically optimizing the transmit pattern during an ultrasonic examination based on changes to a scanning depth parameter. The transmit pattern comprises one or more of pulse sequence, shape of pulses, number of pulses, amplitude of pulses, and transmit frequency. The scanning depth may be a penetration depth or may be defined, for example, by an ROI or range gate. An optimal set of scanning parameters is associated with a probe and each probe may have more than one optimal set of scanning parameters that are each optimized for a particular imaging mode. When the operator changes the scanning depth during a scan, the system automatically optimizes the scanning parameters such as the transmit pattern, receive filter settings and beam-density. If the operator changes a scanning parameter such as the transmit frequency, the system adaptively applies the operator changes to determine the optimal scanning parameters.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. An ultrasound system, comprising:
an ultrasonic probe having transducer elements for transmitting and receiving ultrasound signals to form an ultrasound image;
an optimization module for receiving a first scanning depth based on the ultrasound image, the optimization module determining a first transmit pattern and receive filter settings based on the first scanning depth, the first transmit pattern and the receive filter settings being used to acquire the ultrasound image; and
transmit and receive circuitry for transmitting and receiving the ultrasound signals based on the first transmit pattern.

2. The system of claim 1, wherein the transmit pattern further comprises at least one of pulse sequence, shape of pulses, number of pulses, amplitude of pulses, and transmit frequency.

3. The system of claim 1, wherein the optimization module further selects the first transmit pattern according to one curve within a set of optimal curves representing optimal transmit patterns as a function of scanning depth.

4. The system of claim 1, further comprising a user input for selecting a second scanning depth, the optimization module determining a second transmit pattern based on the second scanning depth, the second transmit pattern being different than the first transmit pattern, and the transmit and receive circuitry transmitting and receiving ultrasound signals based on the second transmit pattern.

5. The system of claim 1, further comprising a user input for changing at least one of a transmit pattern and a transmit frequency, the optimization module identifying a second transmit pattern based on a curve representing an optimal transmit pattern as a function of the first scanning depth.

6. The system of claim 1, further comprising a display having a display resolution, the optimization module further determining a beam-density based at least on the display resolution, the transmit and receive circuitry transmitting and receiving the ultrasound signals based at least on the beam-density.

7. The system of claim 1, wherein the ultrasound system is one of cart-based, portable, hand-held, hand carried and pocket sized.

8. A method for optimizing an ultrasound image, comprising:
scanning with a first transmit pattern based on a first scanning depth to acquire an ultrasound image, the ultrasound image being acquired in a first scanning mode;
receiving a second scanning depth that is different with respect to the first scanning depth; and
selecting a second transmit pattern based on the second scanning depth, the second transmit pattern being different with respect to the first transmit pattern.

9. The method of claim 8, wherein the first and second transmit patterns further comprise at least one of pulse sequence, shape of pulses, number of pulses, amplitude of pulses, and transmit frequency.

10. The method of claim 8, wherein the scanning further comprises defining first and second beam-densities based at least on the first and second scanning depths, respectively.

11. The method of claim 8, wherein the first and second transmit patterns comprise a transmit frequency having first and second transmit frequency values, respectively.

12. The method of claim 8, wherein the first and second transmit patterns comprise a transmit frequency having first and second transmit frequency values, respectively, the method further comprising:
receiving a third frequency value that is different with respect to the second frequency value; and
selecting a third transmit pattern based on the second scanning depth and the third frequency value.

13. The method of claim 8, further comprising:
detecting a desired resolution associated with at least one of an integrated display, an external display, and an operator defined resolution; and
adjusting a beam-density based on the desired resolution.

14. The method of claim 8, further comprising:
detecting a display resolution of a display being used to display the ultrasound image; and
selecting a beam-density of transmitted ultrasound beams based on the display resolution and the second scanning depth.

15. The method of claim 8, further comprising:
selecting a second scanning mode that is different with respect to the first scanning mode; and
selecting a third transmit pattern based at least on the second scanning mode.

16. The method of claim 8, wherein the first and second scanning depths are based on a region of interest located within the ultrasound image.

17. A method for optimizing an ultrasound image, comprising:
selecting a first transmit pattern based on a first scanning depth within an ultrasound image, the first transmit pattern further defining values for at least one of pulse sequence, shape of pulses, number of pulses, amplitude of pulses, and transmit frequency;
selecting a first beam-density based on at least the first scanning depth;
receiving a second scanning depth that is different with respect to the first scanning depth; and
selecting a second transmit pattern and a second beam-density based on the second scanning depth, the second transmit pattern being different with respect to the first transmit pattern.

18. The method of claim 17, further comprising:
receiving a second transmit frequency value; and
selecting a third transmit pattern based on the second transmit frequency value.

19. The method of claim 17, wherein the first and second transmit patterns are based on a set of optimal curves, the set of optimal curves representing optimal transmit patterns based on at least one of scanning depth and transmit frequency.

20. The method of claim 17, wherein the first and second transmit patterns are based on at least one set of optimal curves, the at least one set of optimal curves being associated with at least one of a probe type and a scanning mode associated with the ultrasound image.
